(19) United States
(12) Patent Application Publication
(10) Pub. No.: US 2003/0224420 A1
(43) Pub. Date: Dec. 4, 2003

(54) Method for isolating and measuring proliferation of long-term label retaining cells and stem cells

(76) Inventors: Marc K. Hellerstein, Kensington, CA (US); Sylvia Jeewon Kim, Sunnyvale, CA (US)

Correspondence Address: Michael R. Ward, Morrison & Foerster LLP, 425 Market Street, San Francisco, CA, 94105-2482, US

(21) Appl. No.: 10/407,435

(22) Filed: Apr. 4, 2003

Related U.S. Application Data
(60) Provisional application No. 60/370,599, filed on Apr. 5, 2002.

(51) Int. Cl. C12Q 1/68; G01N 33/53; G01N 33/567; G01N 33/574; C12N 5/08

(52) U.S. Cl. 435/6; 435/7.2; 435/7.23; 435/366

(57) ABSTRACT

This invention relates to a method for separating long-term label retaining cells or stem cells. In particular, this invention relates to a method for separating long-term label retaining cells and/or stem cells from tissues or individuals and for measuring proliferation rates of long-term label retaining cells and stem cells, as well as determining clonal expansion (proliferative history) of cell lineages from the tissues of the individual. The cells may be double-labeled with a cell-lineage marking label and isotopically labeled DNA synthesis precursor prior to physical separation.

### CROSS-REFERENCE TO RELATED APPLICATIONS

This application claims priority to U.S. Provisional Application No. 60/370,599 filed on Apr. 5, 2002, which is hereby incorporated by reference in its entirety.

### FIELD OF THE INVENTION

This invention relates to a method of separating long-term label retaining cells and/or stem cells from non-label retaining cells and/or non-stem cells. In particular, this invention relates to methods of separating stem cells from a tissue or individual, and methods of determining the proliferation rate of long-term label retaining cells and/or stem cells, as well as identifying the clonal expansion (proliferative history) of cell lineages of tissues and individuals.

### BACKGROUND OF THE INVENTION

The number of cell divisions undergone by a particular lineage of cells is of fundamental importance in a number of diseases as well as normal physiologic processes. Cell divisions within a particular lineage, also termed the clonal expansion or proliferative history of a particular cell line, particularly stem cells, influence the risk of cancer (i.e. carcinogenesis), rate of fixation of DNA damage as permanent mutations (i.e., mutagenesis, teratogenesis, carcinogenesis, evolutionary rate), response of T cells to antigenic stimuli (i.e., vaccine efficacy), spermatogenesis (i.e. male fertility), adipogenesis from pre-adipocytes (i.e. body fat accrual), maintenance of epithelial cell populations (i.e. tissue homeostasis), neurogenesis in brain, and other medical conditions and diseases.

Stem cells, of both embryonic and adult types, are of great general interest in a number of areas of biology and medicine, including normal tissue homeostasis, pathogenesis of disease and therapeutics. Epithelial cells line the outside of the body and the hollow tubes (lumens) of the body that communicate with the outside world. Epithelial tissues include the skin, gastrointestinal tract (e.g. colon, small intestine, stomach, esophagus, mouth, exocrine pancreas, etc.), genitourinary tract (e.g. bladder, prostate gland, ovary, uterus, cervix, vagina, urethra), hepatobiliary tract (liver, bile ducts and gall bladder), mammary gland and respiratory system (e.g. bronchi, trachea, alveoli, nasopharynx, etc.). These tissues share the characteristic of constant cell renewal, with old cells being sloughed from the tissue surface into the lumen or the outside world and replaced by recently divided, differentiated epithelial cells. The organization of this cell renewal process shared by all epithelial tissues appears to depend upon epithelial stem cells (or adult stem cells). These epithelial stem cells have two central characteristics: first, the unique capacity for asymmetric cell division (wherein one daughter cell gives rise to a clone of differentiated cells while the other daughter cell remains an undifferentiated, stem cell); and, second, a slow cell division rate. Within each functional unit of an epithelial tissue, such as the crypt of the colon or acinus of the mammary gland, are believed to reside a relatively small number of stem cells or potential stem cells which serve to maintain the flow of differentiated cells to maintain tissue mass and function, in the face of constant cell losses.

Unfortunately, no definitive markers for most stem cells have yet been identified, either on the cell surface or within cells. Part of the problem in identifying markers of “stem-cellness” may be fundamental: stem cells are less differentiated than the daughter cells that surround them in tissues and therefore are notable for what they do not express (e.g. genes and proteins). Because it is more difficult to separate a cell based on what is absent than what is present, stem cells have remained elusive to physical separation. The inability to separate stem cells has held back advances in therapy (e.g. growing stem cells ex vivo for use in the therapeutic regeneration of tissues) as well as diagnostics (e.g. cancer risk based on mitogenic rate or clonal expansion history).

Stem cells are characterized by a long lifespan. A number of long-lived, putative stem-like cells have been documented to exist in numerous epithelial tissues (e.g. skin and intestine) as well as mesenchymal tissues (e.g. bone marrow). These long-term label-retaining cells (LRCs) retain label in their nuclei for a long time after administration of radioactive precursors to DNA (e.g. ³H-thymidine), whereas most cells present in these tissues are free of label within several days due to the rapid replacement of differentiated epithelial cells.

Several lines of evidence support the hypothesis that these long-term label-retaining cells (LRCs) are indeed stem-cells, or are highly enriched for stem cells and, as importantly, are the target cell populations for carcinogens. LRCs are observed in the anatomic location (e.g. the base of the colon crypt or in the central portion of the proliferative unit in skin) expected for epithelial stem cells. LRCs occur as a relatively small fraction of the total differentiated cell population (e.g. 5-10 cells out of a crypt population of 250 cells). Finally, LRCs in skin also exhibit long-term retention of DNA-adducts that are known to be carcinogens, consistent with a direct DNA-binding and mutation-induction in these LRCs.

The retention of nucleotide labels or radioactive labels in DNA of a subpopulation of cells (LRCs) has not allowed for the separation of these cells or direct measurement of how often they divide, however. Genetic or phenotypic characterization of stem cells has therefore not been advanced; ex vivo growth or culturing of separated stem cells for therapeutic purpose has not been achieved; and assessment of cancer risk attributable to the rate of mitogenesis of stem cells has not been possible.

We have recently reported a non-radioactive method for measuring cell proliferation in vivo. Neese, et al., PNAS2002; 99:15345-15350, incorporated herein by reference in its entirety. DNA replication, and thus cell proliferation, is measured based on the incorporation of deuterium from deuterated water (²H₂O) into the deoxyribose (dR) moiety of newly synthesized DNA. This technique has been used after discontinuing deuterium administration to measure the proportion of LRCs present in a population of cells (e.g. T-cells or mammary epithelial cells). Labeling methods have not been used for separating stem cells, or measuring stem cell proliferation rates and stem cell clonal expansion, however.

Indeed at present, there is no reliable technique for externally detecting separating LRCs or stem cells, or for measuring the rate of LRC or stem cell proliferation in tissues and individuals. Thus, there is a tremendous need for reliable methods of identifying and quantifying LRCs and stem cells. Moreover, there is a tremendous need for measuring proliferation rates, and clonal expansion (proliferation history) of cell lineages, in tissues and individuals.

### SUMMARY OF THE INVENTION

In order to meet these needs, the present invention is directed to a method for separating long-term label retaining cells (LRCs) and/or stem cells, and measuring the proliferation rate of LRCs and/or stem cells in tissues and individuals. In one format, the present invention is directed to a method of separating LRCs and/or stem cells based upon their kinetic labeling characteristics. In the method, a cell lineage marking label is administered to a tissue or individual. The cell-lineage marking label is administered at a sufficient quantity and for a sufficient duration to label cells, particularly LRCs and/or stem cells.

The cell-lineage marking label is selected so that it incorporates into dividing cells at sufficient levels to allow external detection and separation of the dividing cells. The cell-lineage marking label is incorporated into the DNA of the cell. The cell-lineage marking label may be a halogenated deoxyribonucleotide (dn) such as bromodeoxyuridine (Brdu) or iododeoxyuridine (Idu). The cell lineage-marking label includes any cell-lineage marking labels that may be externally detected when within the cell.

Once incorporated into a cell, the label is reduced or diluted in that cell by cell division. With each cell division, a fraction of the label is transferred to a daughter cell. The more often the cell divides, the more the label is diluted. In the case of labeled DNA, the proportion of label transferred to the daughter cell is half that of the parent cell due to the semi-conserved replication of DNA. After discontinuing administration of the cell-lineage marker to the tissue or individual, the cells of the tissue or individual are allowed to divide for a sufficient time to permit a first population of non-label retaining cells, which divide more rapidly than a second population of LRCs and stem cells, to reduce the amount or concentration of the cell lineage marking label present in the undivided cells by cell division to levels lower than that of the more slowly dividing LRCs and stem cells.

In one format, the cells of the first population retain no detectable label.

After the non-label retaining cells (i.e. the fast-dividing population of cells) have diluted out the cell lineage marking label by cell division to levels lower than that of the LRC's and/or stem cells, the labeled LRCs and/or stem cells are detected and separated. Such LRCs and stem cells can be identified by antibodies specific to the marking label, and separated by flourescence-activitated cell sorting (FACS) or other methods known in the art, since LRCs and stem cells have greater amounts of the externally detectable label than the non-LRCs and be separated based upon this characteristic.

In one variation, administration of the cell-lineage marking label is discontinued, and the cells are allowed to divide such that the first population of cells contains no detectable label.

In one format, the tissues include colon, breast, small intestine, uterine cervix, prostrate gland, skin, bone marrow, liver heart, skeletal muscle, thymus, thyroid gland, pancreas, bladder, lung, bilary track, ovary, testes, brain, lymphoid tissue, or other stem cell-containing tissues.

Once separated, the LRCs and/or stem cells may be further analyzed and characterized to identify a biochemical marker on the stem cells. The biochemical marker may be subsequently utilized to separate stem cells. In addition, the LRCs and/or stem cells may be analyzed for DNA damage, mutations or other chemical alterations which may result from carcinogen exposure, DNA repair capacity, oxidative damage, mutation risk, or other genotoxic exposures.

In the method of the invention, the methods may be used to identify a chemical agent as genotoxic to LRCs and/or stem cells by administering the chemical agent to the tissue or individual prior to detecting DNA modifications, including DNA chemical modifications, DNA cross-links, DNA mutations, base deletions, base insertions, and intercalations in LRCs and stem cells that have been separated.

The proliferation rate of LRCs and/or stem cells may also be determined after their separation has been achieved.

In one format, the method of the invention further includes administering or contacting the tissue or individual with an isotope labeled DNA synthesis precursor that is incorporated into DNA when a cell of the tissue or individual divides. The isotope labeled DNA synthesis precursor may be administered or contacted with the tissue or individual before, during or after administration of the cell lineage marking label. In the method, the isotope labeled DNA synthesis precursor may be any known DNA synthesis precursor, including, but not limited to, ³H-dT, ²H-glucose, and ²H₂O.

After administering or contacting the organisms or tissue with a quantitative isotope labeled DNA synthesis precursor for sufficient time to permit labeling of DNA in the cells of the tissue or individual, the labeled DNA may be separated by procedures known in the art. Once the DNA is separated, the isotopic enrichment of the DNA or its hydrolysis or degradation products may be measured by mass spectrometry, liquid scintillation counting, gamma counting, nuclear magnetic resonance, and other methods known in the art. Based on this measured isotope enrichment in the separated DNA or its products, the rate of proliferation and turnover of the separated LRCs and/or stem cells can be calculated.

In another format, the one or more hydrolysis products of the DNA (e.g. deoxyribonucleotides) may be measured. The hydrolysis products may also be chemically modified.

The isotopic enrichment may be detected by an analytic method, including mass spectrometry, liquid scintillation counting, gamma counting, and nuclear magnetic resonance spectroscopy. In particular, the isotopic enrichment may be detected by mass spectrometry.

The tissue may be colon, breast, small intestine, uterine cervix, prostate gland, skin, bone marrow, liver, heart, skeletal muscle, thymus, thyroid gland, pancreas, bladder, lung, biliary track, ovary, testes, brain, or lymphoid tissue.

In addition, the method may further include calculating the clonal expansion factor of the cells in the second population.

The methods of the invention find use in detecting factors that influence cancer growth and development; in monitoring treatments for cancer, monitoring influence risks for cancer (i.e. carcinogenesis); monitoring rates of fixation of DNA damage as permanent mutations (i.e., mutagenesis, teratogenesis, carcinogenesis, evolutionary rate); monitoring the response of T cells to antigenic stimuli (i.e., vaccine efficacy), monitoring and designing treatments for spermatogenesis (i.e. male fertility), monitoring adipogenesis from pre-adipocytes (i.e. body fat accrual); monitoring the maintenance of epithelial cell populations (i.e. tissue homeostasis), monitoring pancreatic β-cell clonal expansion, as a marker for the risk of developing diabetes mellitus; monitoring T lymphocyte clonal expansion as a marker of impending immune compromise in progressive lymphopenic disorders such as HIV/AIDS; monitoring bone marrow stem cell clonal expansion as a marker of impaired bone marrow reserve; monitoring cell clonal expansion as a marker or impaired tissue reserve of impending replicative exhaustion and monitoring clonal expansion of transplanted bone marrow cells (graft cells) as a marker of transplant status or of graft vs. host disease.

Kits for practicing the methods disclosed herein are also provided.

### BRIEF DESCRIPTION OF THE DRAWINGS

The invention will be better understood by reference to the following drawings.

FIG. 1 depicts an experimental design for double-labeling a tissue or individual.

FIG. 2 depicts the incorporation of deuterium from water into deoxyribose (dR) of DNA.

FIG. 3 depicts pathways for labeling of DNA in individual cells. Abbreviations used: G, glucose; GNG, gluconeogenesis; P, phosphate; R, ribose; DNPS, de novo purine synthesis pathway; DNNS, de novo nucleotide synthesis pathway; NDP, nucleoside diphosphate; RR, ribonucleoside reductase; dNTP, deoxyribonucleoside triphosphate; DNA, deoxyribonucleic acid; dN, deoxyribonucleosides; dT, thymidine deoxyribonucleoside; BrdU, bromodeoxyuridine.

FIG. 4 depicts body water enrichments during the BrdU delableing/²H₂O labeling period.

FIG. 5 depicts histograms of nuclei from BrdU administered rats analyzed by FACS. Histograms of BrdU positive and negative nuclei from colon epithelial cells are shown. A: Nuclei from rats given BrdU i.p. B: Nuclei from rats given BrdU p.o. BrdU negative regions were established by non-BrdU treated colon epithelial cells (CECs) stained with isotype control antibodies. Note the higher fluorescence of BrdU positive nuclei from BrdU i.p. injected rats compared to BrdU p.o. treated rats. Since the toxicities were observed with the i.p. route, we decided to use p.o.

FIG. 6 depicts histograms of BrdU delabeled CEC. Nuclei from CECs were obtained from 2, 4 and 8 week BrdU delabeled rats and were stained with anti-BrdU FITC antibodies. BrdU negative control was used to establish the BrdU-negative region. A. 1) Histogram of nuclei from 2 week BrdU delabeled rats. 2) Histogram of nuclei from 4 week BrdU delabeled rats. 3) Histogram of nuclei from 8 week BrdU delabeled rats. Since LRCs proliferated slowly during the BrdU labeling period, their fluorescence is not as bright as normal differentiating CEC (FIG. 4). B. Re-analysis by FACS of sorted BrdU positive and BrdU negative nuclei to confirm no overlaps between the two populations.

FIG. 7 depicts average percent of LRC from 0, 2, 4, and 8 weeks delabeled CEC (77±12, 12±11, 7±6.3 and 3.8±1%, respectively). We were able to detect low but distinguishable fraction of LRCs present and further collected them for GC/MS analysis for deuterium enrichments.

FIG. 8 depicts percent of LRC measured by delabeling of ²H₂O. The filled bar shows deuterium enrichment of CEC from in utero labeled rats (n=4). The open bar shows deuterium enrichment of CECs from 2 week ²H₂O delabeled rats (n=3).

FIG. 9 depicts immunohistochemistry of BrdU positive cells from fully BrdU labeled and 4 week delabeled crypts. A) BrdU positive cells on fully BrdU labeled crypts (2 weeks on BrdU). Note the uniform staining with anti-BrdU antibody (brown spots) on the cells lining the inner crypts. B) BrdU positive cells on 4 weeks BrdU delabeled crypts. Note the location of LRCs near the base of the crypt (boxed).

FIG. 10 depicts argentaffin staining for enteroendocrine cells in colonic crypts. Among 33 slides stained, we were able to find enteroendocrine cells in 2 slides only. Furthermore, their locations were not consistent with the LRCs' known location, which is the base of the crypts. Thus, the contamination of enteroendocrine cells in LRC seems to be minimal.

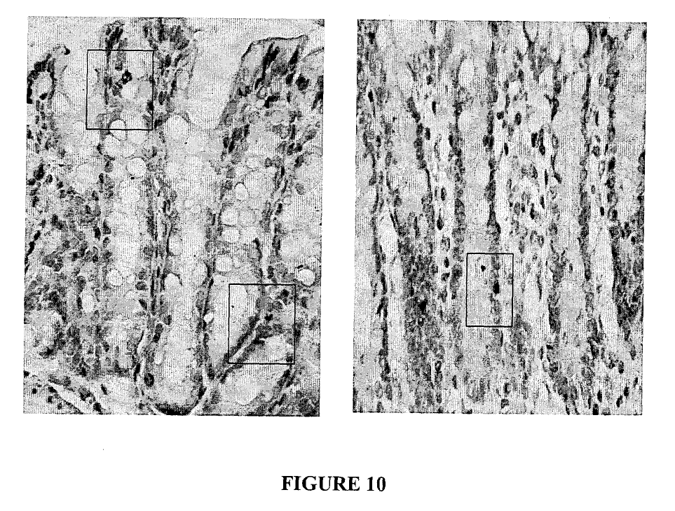

### DETAILED DESCRIPTION OF THE INVENTION

I. General Techniques

The practice of the present invention will employ, unless otherwise indicated, conventional techniques of molecular biology (including recombinant techniques), microbiology, cell biology, biochemistry, immunology, protein kinetics, and mass spectroscopy, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999), all of which are incorporated herein by reference in their entirety. Furthermore, procedures employing commercially available assay kits and reagents will typically be used according to manufacturer-defined protocols unless otherwise noted.

II. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The general techniques and procedures described or referenced herein are generally well understood and commonly employed using conventional methodology by those skilled in the art, such as, for example, Mass isotopomer distribution analysis at eight years: theoretical, analytic and experimental considerations by Hellerstein and Neese (Am J Physiol 276 (Endocrinol Metab. 39) E1146-E1162, 1999). As appropriate, procedures involving the use of commercially available kits and reagents are generally carried out in accordance with manufacturer defined protocols and/or parameters unless otherwise noted.

Stem Cells—Stem cells are slow dividing progenitor cells which have the capacity both to self-renew and to differentiate into mature somatic tissues by forming daughter, non-stem cells. Stem cells, also referred to herein as long-term label retaining cells (LRCs), refer to slow proliferating cells that retain cell-lineage-marking labels and isotope labels. Embryonic stem cells are the archetypal stem cell, being capable of differentiating to form the whole gamut of cell types found in the adult animal. Such stem cells are pluripotential since they are capable of differentiating into many cell types. Examples of stem cells include, but are not limited to, bone marrow stem cells, epidermal stem cells, hematopoietic stem cells, embryonic stem cells, mesenchymal stem cells, epithelial stem cells, gut stem cells, skin stem cells, neural stem cells, liver progenitor cells, endocrine progenitor cells, and lympho-hematopoietic stem cells (which are capable of differentiating into members of the lymphoid, erythroid, and myeloid lineages).

Stem cells can be derived from a variety of sources including, but not limited to, bone marrow, mobilized or unmobilized peripheral blood, umbilical cord blood, fetal liver tissue, other organ tissue, skin, and nerve tissue.

As used herein, stem cells include all long-term label retaining cells (LRCs).

Non-Stem Cells—Non-Stem cells are cells derived from stem cells which have the capacity to differentiate into mature somatic tissues. Non-stem cells divide at detectably faster rates that stem cells. Non-stem cells retain cell lineage marking labels for detectably shorter times than stem cells (LRCs).

Cell-lineage-marking label—A cell lineage marking label is a label that can incorporate into LRCs and stem cells to permit their identification. Cell-lineage marking labels may be incorporated into the DNA of cells or into other components of cells that do not turn over in the absence of cell division. Examples of cell lineage marking labels include halogenated deoxyribonucleotides, such as bromodeoxyuridine (BrdU) and iododeoxyuridine (IdU), that are incorporated into DNA during cell division.

Body water enrichment—Body water enrichment refers to the percentage of total body water that has been labeled upon administration of labeled water.

Genotoxic—Genotoxic and genotoxicity refer to the ability of a chemical or to cause damage to deoxyribonucleotides (or DNA damage). Examples of DNA damage include, but are not limited to, chemical modification of DNA, mutations of DNA, deletions and insertions of bases into DNA, and intercalation into DNA, which are readily known in the art. DNA damage may result in a number of diseases and disorders, including but not limited to, carcinogenesis.

Individual—refers to a vertebrate, preferably a mammal, more preferably a human. Mammals include, but are not limited to, humans, farm animals, sport animals, pets, primates, mice and rats.

Clonal Exhaustion—Clonal exhaustion refers to the elimination of a particular cell type due to activation and proliferation of its stem cell reserve population. An example of clonal exhaustion is the elimination of specific T cells in a patient with HIV/AIDS.

Phenotypic marker—A phenotypic marker is an observable biochemical structure, molecule, function, or behavior associated with a cell, tissue, organism, or individual. Examples of phenotypes include are the physical parts, macromolecules, cell-surface proteins; metabolism, and behaviors of a cell, tissue, organism, or individual.

Clonal Expansion—Clonal expansion refers to cell divisions undergone by a particular cell lineage.

Proliferation history—Proliferation history refers to the history of clonal expansion for a specific cell lineage.

Labeled Water—refers to water labeled with a specific heavy isotope of either hydrogen or oxygen. Specific examples of labeled water include ²H₂0, ³H₂O, and H₂¹⁸O.

Separating—refers to removing one compound from a mixture of compounds. For example, “separating one or more stem cells” refers to removing one or more stem cells from a mixture of one or more stem cells and non-stem cells. Separated stem cells may be accompanied by non-stem cells.

Taking into account these definitions, the present invention is directed to a method of separating stem cells based upon their kinetic labeling characteristics.

Stem Cells

The present invention relates to methods of separating stem cells based on their central kinetic characteristic of long life span, and measuring stem cell proliferation rate, clonal expansion, or proliferative history, or the proliferation rate, clonal expansion, or proliferative history of any dividing cell population by a double-labeling approach.

As discussed above, stem cells are slowly dividing progenitor cells that have the capacity both to self-renew and to differentiate into mature somatic tissues by forming daughter, non-stem cells. Due to their long life and capacity for self-renewal and differentiation, stem cells, particularly epithelial stem cells, are implicated in a number of disease and disorders including carcinogenesis and in a number of normal physiological maintenance and reparative processes, including tissue healing and replenishment.

General Model of Carcinogenesis

The current general model of carcinogenesis involves the sequential accumulation of mutations in genes related to cell cycle control and/or cell death. It is generally held that at least 5-6 somatic mutations are required for the evolution of most neoplastic cells. This process of carcinogenesis therefore occurs over many years, or even decades, and requires persistence or selective advantage for cells that have fixed intermediate numbers of key cell-cycle mutations. Moreover, because each round of mitosis (cell division) increases the likelihood of fixing DNA damage as permanent genetic mutations and of creating new errors in DNA or chromosomes during replication, cell proliferation rate itself (mitogenesis) represents an independent risk factor for cancer, along with DNA damaging agents (mutagenesis).

Epithelial Carcinogenesis—Central Role of Stem Cells

Cancers of epithelial tissues represent the most common cancers of the modern industrial world, including breast, colon, lung, prostate, pancreatic, gastric, esophageal, ovarian, endometrial, and cervical cancers. A seeming paradox is that most epithelial cells reside for a short time in the tissue before dying and/or being sloughed off the tissue surface. Differentiated epithelial cells do not therefore live long enough to accumulate mutations required for a neoplastic clone. The resolution of this paradox is that epithelial stem cells residing in the tissue, dividing occasionally but maintaining a clonal lineage over the course of many years, must represent the target cells for carcinogenic transformation. Epithelial stem cells are therefore believed to be the key to the major cancers of public health concern in the modern world.

Clonal Expansion and Proliferative History of a Cell Lineage

A related issue is the number of cell divisions undergone by any particular lineage of cells. Proliferative history of a cell lineage is of fundamental importance in a number of diseases as well as normal physiologic processes. Cell divisions within a particular lineage, also termed the clonal expansion of a cell line, particularly of stem cells, influence risk for cancer (i.e. carcinogenesis), rate of fixation of DNA damage as permanent mutations (i.e. mutagenesis, teratogenesis, carcinogenesis, evolutionary rate), response of T cells to antigenic stimuli (i.e. vaccine efficacy), spermatogenesis (i.e. male fertility), adipogenesis from pre-adipocytes (i.e. body fat accrual), and maintenance of epithelial cell populations (i.e. tissue homeostasis).

A. Methods of Separating Stem Cells

Stem cells may be obtained by administering a cell lineage marking label, discontinuing administration, detecting cells that retain the cell-lineage marking label, followed by separating cells that retain the cell lineage marking label.

1. Administering a Cell Lineage Marking Label

As a first step, one or more cell-lineage-marking labels are administered to an individual or tissue. The cell-lineage marking label is administered at a sufficient quantity and for a sufficient duration to label cells, particularly stem cells, of the tissue or individual that divide during the period of administration of the label.

The cell-lineage-marking label may be a labeled deoxyribonucleotide (dN). Labeled deoxynucleotides include any labeled deoxyribonucleotides known in the art. The deoxynucletides include any known nucleic acids, including deoxythymidine (dT), deoxyadenosine (dA), deoxycytosine (dC), deoxyguanosine (dG), and deoxyuridine (dU).

Cell-lineage-marking labels may also be halogenated deoxyribonucleotides. Halogenated deoxynucleotides may include any halogenated deoxyribonucleotide, including, but not limited to dT, dA, dC, dG, and dU. Specific examples of halogenated cell-lineage marking label are halogenated deoxyribonucleotide such as bromodeoxyuridine (Brdu), iododeoxyuridine (Idu), and bromodeoxycytidine (BrdC).

The cell lineage marking label may be a radio-labeled nucleotides that may be detected from outside an intact cell. The radiolabel may be any radio-isotope known in the art. These include halogen radio-isotopes, such as Br⁸²-BrdC, Br⁸²-BrdU, Br⁸²-BrdA, Br⁸²-BrdT, and Br⁸²-BrdG. Other radio-labeled nucleotides include tritiated nucleotides, such as ³H-dC, ³H-dG, ³H-dA, ³H-dT, and ³H-dU.

Cell lineage marking labels may also be deuterium labels that may be detected from outside an intact cell. Specific examples include deuterium labeled DNA synthesis precursors such as glucose, and deuterium labeled nucleotides such as ²H-dT, ²H-dA, ²H-dG, ²H-dC, and ²H-dU.

Cell lineage marking labels suitable for use in vivo are prepared in accordance with conventional methods in the art using a physiologically and clinically acceptable solution. Proper solution is dependent upon the route of administration chosen. Suitable routes of administration may, for example, include oral, rectal, transmucosal, transcutaneous, or intestinal administration; parenteral delivery, including intramuscular, subcutaneous, intramedullary injections, as well as intrathecal, direct intraventricular, intravenous, intraperitoneal, intranasal, or intraocular injections.

Cell lineage marking labels may be readily obtained commercially, for example, from Sigma Chemical Company, St. Louis, Mo., United States.

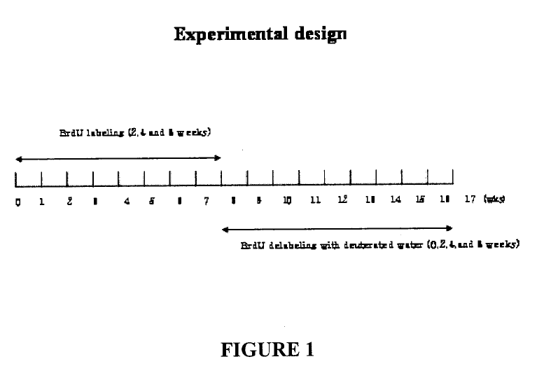

If introduced to an individual, cell-lineage-marking labels may be administered, for example, at 1 mg/ml concentrations in drinking water. If other cell lineage marking labels are utilized, then a non-toxic amount, which is readily determined by those of skill in the art, is administered. The cell-lineage-marking label is administered for a period of time sufficient to be incorporated in the DNA of the organism. Preferably, the cell-lineage-marking label is administered to achieve a steady state concentration in the tissue or organism. For example, the cell-lineage-marking label may be administered for 2, 4, or 8 weeks, or longer, as depicted in FIG. 1.

Alternatively, cell-lineage-marking labels may be administered in a local rather than systemic manner. For example, a cell-lineage-marking label may be administered via injection of directly into a specific tissue, often in a depot or sustained release formulation continuously released in a 1 mg/ml concentration in water. Similarly, the cell-lineage-marking label may be administered until a constant body water enrichment in the DNA of the tissue or individual is achieved.

Administration of the cell-lineage-marking label may be discontinued prior to separating LRCs and/or stem cells.

2. Detecting Cell-Lineage Marking Labels

Once incorporated into a cell, the cell-lineage-marking label is reduced or diluted in that cell by cell division. With each cell division, some of the cell-lineage marking label is transferred to a daughter cell. The faster the cell divides, the faster the cell-lineage marking label is diluted. After discontinuing administration of the cell-lineage marker to the tissue or individual, the cells of the tissue or individual are allowed to divide for a sufficient time to permit the non-stem cells, which divide more rapidly than the stem cells, to reduce the amount of the cell-lineage-marking label by cell division to levels lower than that of the more slowly dividing LRCs and/or stem cells. Discontinuing the administration occurs for sufficient time to form a first population of cells and a second population of cells, where the cells of said second population of cells contain detectably more cell-lineage marking label than the cells of the first population of cells. Administration of the cell-lineage marking label may be for a finite period of time. For example, administration of cell-lineage marking labels may be discontinued for 1 week, 2 weeks, 4 weeks, or 8 weeks.

After the non-stem cells have diluted out the cell-lineage-marking label by cell division to levels lower than that of the LRCs and/or stem cells, the LRCs and/or stem cells may be identified.

Cell-lineage-marking labels may be detected using antibodies that specifically identify the cell-lineage marking labels. An “antibody” (interchangeably used in plural form) is an immunoglobulin molecule capable of specific binding to a target such as a cell-lineage marking label, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity. Antibodies may be monoclonal or polyclonal.

For example, identification of cell-lineage marking labels may be conducted using monclonal antibodies directed to the cell-lineage marking labels BrdU, IdU, or to ²H or ¹³C labeled DNA, or other cellular markers. Anti-BrdU and anti-IdU monoclonal antibodies are available from Pharmingen and Research Diagnostics Inc. Labeling of stem and non-stem cells with BrdU or IdU and the subsequent detection of incorporated BrdU or IdU with specific anti-BrdU or anti-IdU monoclonal antibodies, respectively, may be done by procedures well known in the art.

3. Separating Stem Cells

Cells containing the cell-lineage-marking label are then separated.

Labeled cells can be separated by fluorescence-activated cell sorting (FACS), as well as other methods known in the art. FACS is a technique for separating and sorting cells marked with a fluorescent label, such as a cell lineage marking label, based on how much they fluoresce at a particular wavelength. FACS methods are described, for example, Flow Cytometry: A Practical Approach. M. G. Ormerod, Editor, 2nd Edition, IRL Press, Oxford 1994, incorporated herein by reference in its entirety.

In another embodiment, a radioisotope-labeled cells may be observed externally to the cell by methods such as liquid scintillation or gamma-counting. Isotopically labeled cells may also be observed mass-based separation techniques, such as centrifugation.

The LRCs and/or stem cells can be separated from tissues including the colon, breast, small intestine, uterine cervix, prostate gland, skin, bone marrow, liver, heart, skeletal muscle, thymus, thyroid gland, pancreas, bladder, lung, biliary track, ovary, testes, brain, lymphoid tissue, or other tissues potentially containing stem cell populations.

The label incorporation may be achieved by other methods. For example, population of cells that transiently expresses green fluorescent protein (GFP) is retained in stem cells, but is diluted with each cell division. GFP is retained in stem cells, but is lost in rapidly dividing non-LRCs. Transiently expressed proteins such as GFP may be used as cell-lineage marking labels, or may be used for the purposes of the invention disclosed herein.

B. Measuring the Rate of Stem Cell Proliferation

In addition to separating LRCs and/or stem cells, the proliferation rate of LRCs and/or stem cells may be determined. The proliferation rate of LRCs and stem cells is implicated in the risk for cancer (i.e. carcinogenesis), rate of fixation of DNA damage as permanent mutations (i.e., mutagenesis, feratogenesis, carcinogenesis, evolutionary rate) response of T cells to antigenic stimuli (i.e., vaccine efficacy), spermatogenesis (i.e. male fertility), adipogenesis from pre-adipocytes (i.e. body fat accrual), maintenance of epithelial cell populations (i.e. tissue homeostasis), and other medical conditions and diseases.

The involvement of cancerous epithelial cells, which lead to the formation of solid tumors in humans, in such organs as the lungs, breast, skin, mouth, and colon are known as carcinomas. Cancers involving human epithelial cells come from solid tumors of the breast, lung, stomach, liver, uterus, colon, skin, mouth and uterine cervix can form. Adenocarcinomas from secretory tissue and squamous carcinomas from protective linings are the two basic categories of carcinomas. Epithelial cell based cancers proliferate rapidly respecting no cellular boundaries. Since many cancers originate in epithelial cells, it is of tremendous interest to be able to measure the growth rates of epithelial LRCs and/or stem cells.

The proliferation rates of LRCs and/or stem cells can be determined by a double-labeling method. Both cell-lineage-marking labels and different isotopically labeled precursors for DNA synthesis are administered to the cells. After separating LRCs and/or stem cells by detecting the long term cell-lineage-marking label retaining cells, the isotopic enrichment of the DNA is measured. The proliferation rate, clonal expansion, and proliferation history may then determined or monitored.

1. Administering a Cell-Lineage Marking Label

As a first label, one or more cell-lineage-marking labels are administered to a tissue or individual, as described above. Administration may be discontinued.

2. Administering an Isotopically Labeled DNA Synthesis Precursor

As a second label, one or more isotopically labeled DNA synthesis precursors is administered to the tissue or individual. The isotopically labeled DNA synthesis precursor is incorporated into deoxyribonucleotides that combine to form new DNA when a cell divides. The isotopically labeled DNA synthesis precursor may be administered before, during or after the cell-lineage-marking label. The isotopically labeled DNA synthesis precursor may be a stable isotope or radioisotope. Isotope labels that can be used include, but are not limited to, ²H, ¹³C, ¹⁵N, ¹⁸O, ³H, ¹⁴C, ³⁵S, ³²P, ¹²⁵I, ¹³¹I, or other isotopes of elements present in organic systems. In one embodiment, the isotope label is ²H.

The DNA synthesis precursor may be any DNA synthesis precursor known in the art.

The DNA synthesis precursor may be CO₂, NH₃, urea, O₂, glucose, lactate, H₂O, acetate, ketone bodies and fatty acids, glycine, succinate or other amino acids, and phosphate.

The isotopically labeled DNA synthesis precursor may also include one or more nucleoside residues. The DNA synthesis precursor may also be one or more components of nucleoside residues. Glycine, aspartate, glutamine, and tetryhydrofolate, for example, may be used as precursor molecules of purine rings. Carbamyl phosphate and aspartate, for example, may be used as precursor molecules of pyrimidine rings. Adenine, adenosine, guanine, guanosine, cytidine, cytosine, thymine, or thymidine may be given as a DNA synthesis precursor. All isotope labeled DNA synthesis precursors may be purchased commercially, for example, from Cambridge Isotope Labs (Andover, Mass.).

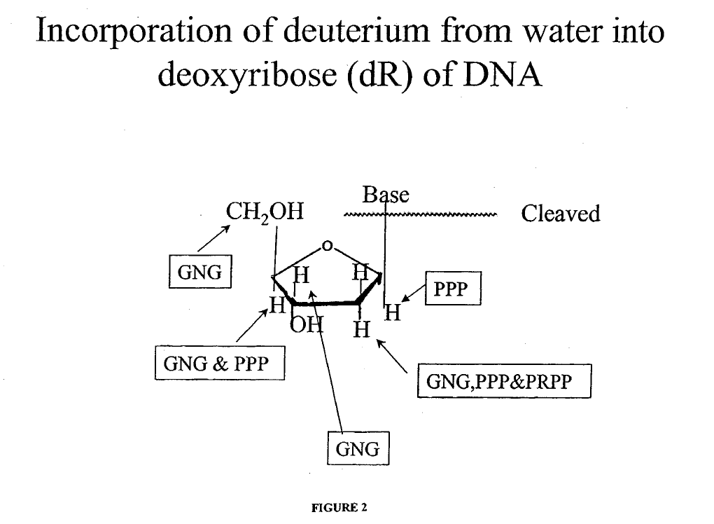

The DNA synthesis precursor may be water. The hydrogen atoms on C—H bonds of polynucleotides, polynucleosides, and nucleotide or nucleosides may be used to measure DNA synthesis from ²H₂O. C—H bonds undergo exchange from H₂O into deoxyribonucleotides in the cell. The presence of ²H-label in C—H bonds of polynucleotides, nucleosides, and nucleotide or nucleoside precursors, after ²H₂O administration therefore means that the DNA was synthesized during this period. The degree of labeling present may be determined experimentally, or assumed based on the number of labeling sites in DNA. For example, FIG. 2 depicts the incorporation of deuterium from water into deoxyribose of DNA.

Hydrogen atoms from body water may be incorporated into free nucleosides or polynucleotides. ²H or ³H from labeled water can enter these molecules through the reactions of intermediary metabolism.

One of skill in the art will recognize that labeled hydrogen atoms from body water may be incorporated into other polynucleotides, nucleotides, or nucleosides via various biochemical pathways. For example, glycine, aspartate, glutamine, and tetryhydrofolate, which are known precursors of purine rings. Carbamyl phosphate and aspartate, for example, are known precursors of pyrimidine rings. Ribose and ribose phosphate, and their synthesis pathways, are known precursors of DNA synthesis.

Oxygen atoms (H₂¹⁸O) may also be incorporated into polynucleotides, nucleotides, or nucleosides through enzyme-catalyzed biochemical reactions, including those listed above. Oxygen atoms from ¹⁸O₂ may also be incorporated into nucleotides by oxidative reactions, including non-enzymatic oxidation reactions (including oxidative damage, such as formation of 8-oxo-guanine and other oxidized bases or nucleotides).

Isotope labeled DNA synthesis precursors may also include isotope labeled amino acids. Isotope labeled amino acids include, but are not limited to, ¹³C-lysine, ¹⁵N-histidine, ²H₅-histidine, other ¹⁵N-labeled or ¹³C-labeled amino acids, and deuterated amino acids.

In particular, the isotope labeled DNA synthesis precursor may be ²H-glucose or ²H₂O, as described in U.S. Pat. Nos. 5,910,403 and 6,010,846 both of which are hereby incorporated by reference in their entirety.

Administration of isotope labeled DNA synthesis precursor may be discontinued prior to detecting cell-lineage marking labels or measuring the isotopic enrichment of DNA.

3. Detecting Cell-Lineage Marking Labels

One or more cells that contain the one or more stem-cell-marking labels may then be separated, as described above.

4 Measuring Isotopic Enrichment of DNA

The level of incorporation of stable isotope label into the DNA of cells is determined by obtaining the DNA from a cell population of interest and analyzing for isotope content a chemical portion of the DNA molecule that is able to incorporate label from in the isotopically labeled DNA synthesis precursor as described above, using standard analytical techniques. Examples of techniques include, for example, mass spectroscopy, and nuclear magnetic resonance, and liquid scintillation counting. Methods of sample preparation will depend on the particular analytical techniques used to detect the presence of the isotope label, and will be apparent to those of skill in the art.

The DNA of cells containing the cell-lineage-marking label may be partially purified or isolated, from the cells. DNA may be obtained from the cells by any method known in the art, such as those described in Sambrook, J. et al, (1989, Molecular Cloning: A Laboratory Manual, Cold Spring Harbor Press, Plainview, N.Y., which is hereby incorporated by reference in its entirety. The actual method of DNA isolation will depend on the particular cell type, and will be readily apparent to those of skill in the art.

The DNA may be hydrolyzed to deoxyribonucleosides using standard methods of hydrolysis as are well-known in the art. For example, the DNA can be hydrolyzed enzymatically, such as for example with nucleases or phosphatases, or non-enzymatically with acids, bases or other methods of chemical hydrolysis. The hydrolysis products may optionally be measured following either partial purification or isolation by any known separation method, as described previously.

Incorporation of one or more isotope labels in DNA may be detected by various methods such as mass spectrometry (including but not limited to gas chromatography-mass spectrometry (GC-MS), isotope-ratio mass spectrometry, GC-combustion-isotope ratio-MS, GC-pyrolysis-isotope ratio-MS, liquid chromatography-MS, electrospray ionization-MS, matrix assisted laser desorption-time of flight-MS, Fourier-transform-ion-cyclotron-resonance-MS, cycloidal-MS), nuclear magnetic resonance (NMR), and liquid scintillation counting.

a. Mass Spectrometry

In a preferred embodiment of the invention, the presence of the isotope label is detected by mass spectrometry as described, for example, in U.S. Pat. Nos. 5,910,403 and 6,010,846.

Mass spectrometers convert components of a sample into rapidly moving gaseous ions and separate them on the basis of their mass-to-charge ratios. The distributions of isotopes or isotopologues of ions, or ion fragments, may thus be used to measure the isotopic enrichment in cellular DNA.

Generally, mass spectrometers include an ionization means and a mass analyzer. A number of different types of mass analyzers are known in the art. These include, but are not limited to, magnetic sector analyzers, electrostatic analyzers, quadrapoles, ion traps, time of flight mass analyzers, and fourier transform analyzers. In addition, two or more mass analyzers may be coupled (MS/MS) first to separate precursor ions, then to separate and measure gas phase fragment ions.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS is used to measure mass isotopomer abundances of organic molecules, hydrogen-labeled isotope incorporation from labeled water is amplified 3 to 7-fold, depending on the number of hydrogen atoms incorporated into the organic molecule from labeled water.

In another embodiment, the isotope labeled DNA may be partially purified, or optionally isolated, prior to mass spectral analysis. Furthermore, hydrolysis or degradation products of isotopically labeled DNA may be purified.

In another embodiment, isotope enrichments of isotope labeled DNA after hydrolysis is measured by gas chromatography-mass spectrometry.

Deoxyribonucleosides may be prepared for mass spectrometric analysis using standard techniques (such as synthesis of trimethylsilyl, methyl, acetyl, etc. derivatives; direct injection for liquid chromatography; and direct probe sample introduction) and the level of incorporation of label into the deoxyribonucleosides determined.

The mass spectrometric analysis is of fragment potentially containing stable isotope label introduced from endogenous labeling pathway. For example, the m/z 467-469 fragment of the deoxyadenosine or the m/z 557 and 559 fragment of the deoxyguanosine mass spectrum, which contain the intact deoxyribose ring, could be analyzed after 6,6 ²H₂ glucose administration, using a gas chromatograph/mass spectrometer under electron impact ionization and selected ion recording mode.

b. Liquid Scintillation

Radioactive isotopes may be observed using a liquid scintillation counter. Radioactive isotopes such as ³H emit radiation that is detected by a liquid scintillation detector. The detector converts the radiation into an electrical signal, which is amplified. Accordingly, the number of radioactive isotopes in a cell or in DNA may be measured.

In one embodiment, the radioisotope-enrichment value in the isotopically labeled DNA may be measured directly by liquid scintillation. In a further embodiment, the radio-isotope may be ³H.

In another embodiment, the isotope labeled DNA or components thereof may be partially purified, or optionally isolated, and subsequently measured by liquid scintillation counting.

5. Calculating the Proliferation Rate and Clonal Expansion

In each of the above embodiments the biosynthesis or breakdown rate of the measured LRCs and/or stem cells may be calculated by application of the precursor-product relationship using either isotope enrichment values or asymptotic isotope enrichment. Alternatively, the biosynthesis or breakdown rate may be calculated using an exponential decay curve by application of exponential or other die-away kinetic models.

The proliferation rate, or “turnover rate,” may be calculated by mass isotopomer analysis, by hand or via an algorithm. In brief, the fractional replacement (%) of newly synthesized DNA at each time point during administration of the isotopically labeled DNA synthesis precursor is determined from the excess abundances.

The fractional replacement (f) of newly divided cells in at each time point during administration of one or more isotopically labeled DNA synthesis precursors may be determined as follows:

$$\begin{matrix}\begin{matrix}{EM}_{1} = \frac{Abundance of {M}_{+ 1 } ( sample )}{Abundance of {M}_{+ 0} + {M}_{+ 1 } ( sample )} - \\ \frac{Abundance of {M}_{+ 1 } ( unlabeled standard )}{Abundance of {M}_{+ 0} + {M}_{+ 1 } ( unlabeled standard )}\end{matrix} \\ f ( fraction of new cells ) = \frac{{EM}_{1} ( cell sample )}{{EM}_{1} ( fully replaced cells )} \times 100\end{matrix}$$

where, M₊₀, parent mass isotopomer of derivatized dR; M₊₁, mass plus one isotopomer of derivatized dR; EM₁, excess abundance of M₊₁ mass isotopomer. For instance, colonocytes are known to be fully replaced with new cells in 6-8 days in rodents. Tissue or individuals may be maintained on one or more isotopically labeled DNA synthesis precursors for a period of time, for example 2-3 weeks, to ensure complete turnover and the enrichments of their fully replaced cells were used as a comparison (denominator) or asymptotic value.

The replacement rate constant (k) may be calculated by:

k=−ln(1−f)/t

where k=rate constant (d⁻¹) and t=labeling period (days)

t_{1/2}(half-life)=0.693/k

One example of mass isotopomer analysis is Mass Isotopomer Distribution Analysis (MIDA), or combinatorial analysis. These algorithm are discussed in a number of different sources known to one skilled in the art. Specifically, the MIDA calculation methods are the subject of U.S. Pat. No. 5,336,686, incorporated herein by reference. The method is further discussed by Hellerstein and Neese (1999), as well as Chinkes, et al. (1996), Kelleher and Masterson (1992), and Macallan et al. (1998), and Neese (2001) which are hereby incorporated by reference in their entirety.

In addition to the above-cited references, calculation software implementing the method is publicly available from Professor Marc Hellerstein, University of California, Berkeley.

The mean clonal expansion factor may also be used to compare proliferation rates among different populations. The mean clonal expansion (C.E.) factor is defined by the following equation:

C.E.=ln(1−f)⁻¹.

The clonal expansion factor is used to compare proliferation rates or turnover rates between different populations of cells. The rate constant k equals CE/t, where t is the labeling period. Thus, k is a rate constant, fractional turnover, or proliferation rate measured per time point.

The clonal expansion factor is used to calculate proliferation history or a cell lineage. Clonal expansion defines how much a population of cells has expanded exponentially in a certain period of time. Thus, the clonal expansion factor may be used to compare “proliferation rates or turnover rates” between different populations. For example, some cells may turn over at a 20% rate per day, while others turn over at a rate of 0.7% per day. The clonal expansion factor of different populations of cells may have different clonal expansion factors, which may be compared.

C. Determining the Presence of Stem Cells Using Isotope-Labeled DNA Synthesis Precursor Molecules

The presence of LRCs and/or stem cells may be determined using only isotopically labeled DNA synthesis precursors of DNA, using a one-label approach. In this method, an isotopically labeled DNA synthesis precursor is administered to a tissue or individual, as described above. Optionally, a stable DNA synthesis precursor enrichment level is maintained. When DNA synthesis precursor is discontinued and isotopic enrichment of deoxyribonucleic acid in the tissue or individual is measured in different cells, cells that retain elevated levels of the isotope are considered LRCs and/or stem cells. By monitoring the same tissue over time, the stem cell proliferation rate and clonal expansion may be measured, as described above.

This one-label approach is in contrast to the double-label approach described above, and does not allow separation of LRCs and/or stem cells. It only allows dtermination, of the presence, absence, or abundance of LRCs and/or stem cells in a tissue.

Utility of Stem Cell Separation and Proliferation Rate Measurement

The methods of the invention find use in detecting and quantifying cancer growth and development; in monitoring treatments for cancer, monitoring influence risks for cancer (i.e. carcinogenesis), monitoring rates of fixation of DNA damage as permanent mutations (i.e., mutagenesis, feratogenesis, carcinogenesis, evolutionary rate), monitoring the response of T cells to antigenic stimuli (i.e., vaccine efficacy), monitoring and designing treatments for spermatogenesis (i.e. male fertility); adipogenesis from pre-adipocytes (i.e. body fat accrual); monitoring the maintenance of epithelial cell populations (i.e. tissue homeostasis); monitoring pancreatic β-cell clonal expansion, as a marker for the risk of developing diabetes mellitus; monitoring T lymphocyte clonal expansion as a marker of impending immune compromise in progressive lymphopenic disorders such as HIV/AIDS; monitoring bone marrow stem cell clonal expansion as a marker of impaired bone marrow reserve; monitoring stem cell clonal expansion as a marker of impaired tissue reserve of impending replicative exhaustion; and monitoring clonal expansion of transplanted bone marrow cells (graft cells) as a marker of transplant status or of graft vs. host disease (GVHD).

Once the LRCs and/or stem cells are separated, they may be characterized by procedures well known in the art. For example, the LRCs and/or stem cells may be analyzed to determine if their DNA is in any way modified by procedures exemplified in Technologies for Detection of DNA Damage and Mutations edited by G. P. Pfeifer, Plenum Press, 1996, hereby incorporated by reference in its entirety. DNA modifications may result from carcinogen exposure, DNA repair capacity, oxidative damage, mutation risk, or other potentially genotoxic exposures, the effects of which can be measured.

The methods described herein may be used to identify disease or disorder. For example, the methods may be used to identify and quantify carcinogenesis. Stem cells are important in colon carcinogenesis since they may be the earliest progenitors of carcinoma, for example, see Bjerknes, M. (1996), Expansion of mutant stem cell populations in the human colon, J Theor Biol 178, 381-385, which is incorporated herein by reference in its entirety. Due to their characteristics such as slow proliferation rates, longer life span in the tissue, high proliferative potential and pluripotency, colon stem cells are thought to be the major cell type at risk for accumulating mutations leading to carcinoma. For example, see Bach, S. P., Renehan, A. G., and Potten, C. S. (2000), Stem cells: the intestinal stem cell as a paradigm, Carcinogenesis 21, 469-476, which is incorporated herein by reference in its entirety. In contrast, other normal differentiating cells are short-lived. When mutated stem cells undergo clonal expansion, the risk for subsequent mutations in the daughter cells increases and renders cells more susceptible to initiation.

The methods described herein may also be used to identify other disease and disorders. For example, the methods may be used to identify risk of, progression toward, or efficacy of treatment against diabetes mellitus, by use of pancreatic β-cells. The methods may also be used to identify immune compromise, for example by measuring T lymphocyte progenitors to identify progressive lymphopenic disorders, such as AIDS. The methods may also be used to measure the onset of transplant rejection by measuring T cell proliferation to identify graft vs. host disease (GVHD).

LRCs and/or stem cells having altered proliferation rates may be used to identify DNA modifications. Examples of DNA modifications include, but are not limited to, chemical modification of DNA, crosslinking DNA, mutations of DNA, deletions and insertions of bases into DNA, and intercalation into DNA, which are readily known in the art. Alternatively, the methods may be used to evaluate the genotoxicity of a putative genotoxic agent. The effects of a putative genotoxic agent may be evaluated by separating LRCs and/or stem cells, or measuring the proliferation rate of the LRCs and/or stem cells, and correlating the LRCs and/or stem cells or proliferation rate to DNA modifications by methods known in the art.

The methods described herein may also be used to identify stem cell phenotypic markers. Phenotypic markers include any observable biochemical structure, molecule, function, or behavior associated with LRCs and/or stem cells. Once the LRCs and/or stem cells are separated, they may be investigated to determine if there are any additional phenotypic markers. Alternatively, phenotypic markers of LRCs and/or stem cells having altered proliferation rates may be investigated to identify phenotypic markers.

The methods described herein may also be used to identify therapeutic compounds. For example, a putative therapeutic compound may be administered to a tissue or individual prior to, or concurrently with, administration of a stem-cell-lineage-marking label and/or an isotopically labeled DNA synthesis precursor. The effect of the putative therapeutic compound may then be monitored. Putative therapeutic compounds may be chemicals or pharmaceuticals. Putative therapeutic compounds may also be dietary factors, including soy derived products such as genistein and lunasin, brassica-derived products, and anti-oxidants such as vitamin C, vitamin E, and vitamin A. LRCs and/or stem cells of a treated tissue or individual may be compared to an untreated tissue or individual to identify effects of the putative therapeutic compounds.

The methods may also used to identify compounds that stimulate stem cell proliferation. For example, after administration of a putative lymphocyte co-stimulator, T cells in the tissue or individual may be monitored to observe the effects of the co-stimulator. Alternatively, clonal or replicative exhaustion may be measured.

The invention will be better understood by reference to the following non-limiting example.

### EXAMPLE

A double-labeling approach was developed using bromodeoxyuridine (BrdU), a thymidine analogue, as a marker for label retaining cells (LRCs) and ²H₂O to determine their proliferation rates using gas chromatography-mass spectrometer (GC-MS). A schematic diagram of the labeling experiments is presented in FIG. 1.

Animals

Fisher 344 rats (male, four weeks old at the beginning of studies, Simonsen, San Jose, Calif.) were used. All procedures were approved by the UC Berkeley Office of Laboratory Animal Care. Housing was 3 rats per cage. Diet was Purina rat chow, provided ad-libitum. A twelve hour light:dark cycle was maintained. At each measurement time point, rats were euthanized by carbon dioxide asphyxiation. There were no significant differences in the body weights of animals while they were given BrdU in drinking water compared to control animals on non-BrdU water. The animals were divided into three groups, receiving 2,4, and 8 weeks of BrdU in drinking water, respectively. The rats were then administered 8% ²H₂O and sacrificed after 0, 2, 4 and 8 weeks of the BrdU-delabeling/²H₂O labeling period (4-9 rats per each delabeling time point) (FIG. 1).

BrdU Administration

For BrdU labeling, rats received BrdU in drinking water at 1 mg/ml. Glucose (1 g/100 ml) was added to the water to mask the taste of BrdU. BrdU was added to the water, then the bottle was shaken. Next, glucose was added and the bottle was vigorously shaken to dissolve BrdU completely. It took approximately 1-2 min for BrdU to dissolve completely. It was important to prepare BrdU drinking water freshly everyday during the feeding period.

²H₂O Administration

Rats were primed with 99.9% ²H₂O i.p. at the start of the ²H₂O labeling period. The volume injected was 4% of the estimated body water volume. Then they were maintained on 8% ²H₂O enriched drinking water ad libitum for up to 8 weeks (19).

Body Water Enrichment

FIG. 2 shows a diagram illustrating the incorporation of deuterium from water into deoxyribose (dR) of DNA. Body water enrichments were measured by a gas chromatographic/mass spectrometer (GC/MS) technique that we have described previously.

Colon Epithelial Cell (CEC) Separation

CECs were separated from the base of the crypt where LRCs are presumed to reside, and the nuclei were fixed and stained for flow cytometer using monoclonal anti-BrdU FITC antibodies.

Colon was excised fresh at necropsy. Feces were removed with 0.015M NaCl solution with 0.001M dithiothreitol (DTT) and the colon was placed in cold, 1.6% Joklik's modified medium (JMMEM, Gibco Invitrogen, Grand Island, N.Y.) for 120 minutes. CECs from different proliferative fractions of the crypt were separated sequentially using the nonenzymatic, mechanical dissociation method described elsewhere. CEC from different crypt fractions (basal proliferative fraction and top mature fraction) were collected by incubating the colon sac filled with PBS buffers containing chelating agents. The tissue was shaken in a 37° C. water bath for 20 minutes, then 15 minutes and 30 minutes for collecting CECs from top, middle and the basal fractions, respectively. The middle fraction (transitional fraction) was discarded. The collected CECs were then separated from intraepithelial lymphocytes and other contaminants by applying to a discontinuous Percoll (Amersham Biosciences, Uppsala, Sweden) gradient centrifugation, with 45% Percoll overlaid on 75% Percoll. CEC from the proliferative fraction (the base of the crypts) were collected and used for FACS and GC/MS analysis since LRCs are known to be located at this region. The CECs from the mature (top) fractions from the rats that were given ²H₂O for 2-3 weeks were used as fully turned over CECs to represent the maximal ²H labeling in DNA. A part of the distal and proximal colon was used for immunohistochemistry.

Flow Cytometric Analysis of BrdU Labeled Cells

The staining protocol was as described elsewhere, and was modified for CECs. The CECs were separated from colon (as described above) and filtered through a Falcon tube 2235. The cells were then fixed with ice cold 95% ethanol while vortexing, centrifugated at 800 g for 3 minutes and washed in cold PBS (without Ca⁺⁺ and Mg⁺⁺) once. Because the CECs showed a tendency to aggregate, nuclei were extracted from the cells for separation by flow cytometry. Two million cells were washed of ethanol in 150 ul of PBS, incubated in 1% NP 40 (Sigma) solution in the refrigerator for 30 minutes, centrifugated at 10,000 g, and the pellet was collected and washed in PBS. Two million CEC nuclei were added to each well in 96 well plates (round bottom), then 150 ul of 1% paraformaldehyde with 0.01% Tween 20 in PBS was added for additional fixation and permeabilization. The cells were kept at room temperature for 30 minutes then at 4° C. for 15 minutes, prior to centrifugation at 800 g for 3 minutes and washing in cold PBS once. Next, 150 μl of DNase I (50 kunits/ml) was added at room temperature for 15 minutes to allow denaturation of nuclear DNA. The cells were then washed with PBS once and 50 μp of FITC (fluoresecin isothiocyanate)-conjugated anti-BrdU monoclonal antibody (Becton Dickinson, Palo Alto, Calif.) was added with 20 μl of PBS containing 0.1% Tween 20. For negative controls, an equal concentration of mouse IgG1 FITC isotype control antibodies (Becton Dickinson, Cat 349041) were used to correct for background fluorescence. The cells were stained over night at 4° C. and washed two times with cold PBS the next day, then filtered with a Falcon tube 2235 before analyzing. The cells were sorted with a Beckman-Coulter EPICS Elite cell sorter equipped With an argon ion 15 mW air cooled laser, a He—Ne 15 mW air cooled laser and a water cooled 5W argon ion laser.

Cell Proliferation Assay

A) Isolation of Deoxyadenosine (dA) From DNA and Derivatization of Deoxyribose (dR)

LRCs were collected by FACS from proliferative CECs obtained from rats and their DNA was isolated using a Qiagen kit (Qiagen, Valencia, Calif.). DNA was hydrolyzed and dA was isolated as described previously. The isolated dA was cleaved of the base moiety to obtain dR. The pentose-tetraacetate (PTA) derivative of dR was prepared for GC/MS analysis. GC/MS analysis was by methane CI, using a 30-m DB-225 column (0.25-mm id, 0.25-μm film thickness, J & W Scientific, Folsom, Calif.), with selected ion monitoring of m/z 245 and 246.

B) Calculations

The % of newly synthesized cells calculated by comparison to a fully turned over CECs.

The fractional replacement (%) of newly synthesized DNA at each time point during ²H₂O administration was determined by the following equation: M₀, parent mass isotopomer of derivatized dR; M₊₁, mass plus one isotopomer of derivatized dR; EM₁, ratio of M₁ molecules over (M₀+M₊₁) abundances corrected for a natural abundance of M₁-mass isotopomer, i.e. excess abundance of M₊₁ mass isotopomer.

The fractional replacement (f) of newly divided cells in colon at each time point during ²H₂O administration was determined as follows:

$$\begin{matrix}\begin{matrix}{EM}_{1} = \frac{Abundance of {M}_{+ 1 } ( sample )}{Abundance of {M}_{+ 0} + {M}_{+ 1 } ( sample )} - \\ \frac{Abundance of {M}_{+ 1 } ( unlabeled standard )}{Abundance of {M}_{+ 0} + {M}_{+ 1 } ( unlabeled standard )}\end{matrix} \\ f ( fraction of new cells ) = \frac{{EM}_{1} ( colon cell sample )}{{EM}_{1} ( fully replaced colon cells )} \times 100\end{matrix}$$

where, M₊₀, parent mass isotopomer of derivatized dR; M₊₁, mass plus one isotopomer of derivatized dR; EM₁, excess abundance of M₊₁ mass isotopomer. Colonocytes are known to be fully replaced with new cells in 6-8 days in rodents.

The replacement rate constant (k) was calculated as described previously:

k=−ln(1−f)/t

where k=rate constant (d⁻¹) and t=labeling period (days)

t_{1/2}(half-life)=0.693/k

Rats were therefore maintained on ²H₂O for 2-3 weeks to ensure complete turnover and the enrichments of their CEC were used as a comparison (denominator) or asymptotic value (18-20).

The body water ²H₂O enrichments during ²H₂O administration are shown in Table 1.

TABLE 1
Body Water ²H₂O enrichments during ²H₂O administration

| | ²H₂O |
|---|---|
| Week | Enrichment |
| 1 | 4.29 |
| 3 | 3.93 |
| 4 | 4.30 |

Proliferation rates of LRC (putative stem cells) in colon were then calculated:

f(% New DNA)=0.33%=0.99%/day

t_{1/2}=77-210 days

BrdU Immunohistochemistry

The distal end of the colon (1 cm) was cut and fixed in 10% PBS buffered formalin overnight. The fixed tissue was embedded in paraffin and sliced into 5 micron sections for mounting onto slides (Histotec laboratory, Hayward, Calif.). Slides were warmed to 56-58° C. for 30 minutes and deparaffinized with Hemo-D clearing agent (Fisher Scientific, Pittsburg, Pa.). Tissue was rehydrated using gradations of 100% to 70% ethanol. Slides were incubated at 40° C. in 2N HCl. Endogenous peroxidase activity was quenched with 3% H₂O₂ for 15 min. Non-specific binding was blocked using a horse serum blocking solution (Vectastain ABC Elite kit, Vector, Burlingame, Calif.). Slides were then incubated with anti-BrdU monoclonal antibody (1:50 dilution in PBS) (Becton-Dickinson, San Jose, Calif.) in a humid chamber at 4° C. overnight, followed by a biotinylated secondary antibody (Vectastain ABC kit, Vector). Detection was performed using streptavidin (Vectastain ABC kit, Vector) and color was developed using 3,3′-diaminobenzidine tetrahydrochloride (DAB) (Sigma) for 7-15 min. Cells were counterstained with Mayer's Hematoxylin solution (Sigma) and slides were preserved with Permount (Fisher Scientific). Labeling index [(no. of BrdU positive cells)/total no. of cells counted] was determined using a Zeiss microscope with a video camera and a monitor. For the colon, at least 10-15 well defined crypts (1000-1500 total cells) from each sample were counted. All cell counts were carried out independently by two observers blinded to the study design.

Argentaffin Staining

Enteroendocrine cells are specifically stained with argentaffin stainings. Colon tissue was fixed in the same way as for BrdU immunohistochemistry. Fixed tissues were stained by Fontana-Masson argentaffin reaction method. Enteroendocrine cells appear black on the pink background of cytoplasm.

Results

Body Weights and Water Consumption During BrdU Administration

Body weight in the BrdU (p.o.) administered rats increased normally as compared to control rats (not shown). The amount of BrdU water consumed per day was 48±12 ml/rat.

Stability of Deuterium Enrichments in Body Water During ²H₂O Labeling Period

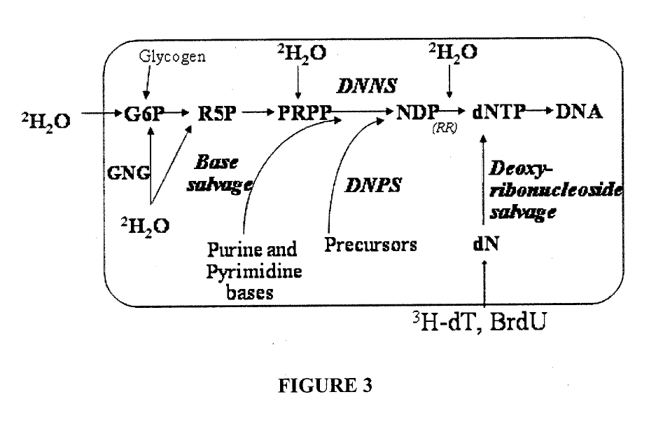

Body water ²H₂O enrichments were measured from plasma obtained from rats sacrificed at each time point. Achievement of plateau ²H enrichments in body water during long-term ²H₂O administration is desirable because it signifies a stable DNA synthesis precursor enrichment for labeling of DNA. Body water enrichment from rats that were on ²H₂O for 2-8 weeks reached expected values and remained at those plateau levels (3.9-4.3%) throughout the study (FIG. 3).

Histogram of CECs From BrdU Administered Rats p.o. and i.p.

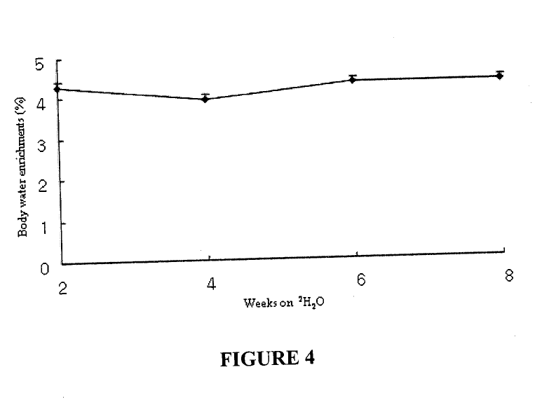

The histograms of CECs from rats administered with BrdU p.o. (in drinking water) vs i.p. is shown in FIG. 4. Although the CEC from repeated i.p. injected (once a day for 8 days) rats resulted in more BrdU incorporation and thus higher fluorescence, we administered BrdU p.o. since i.p. treatment caused toxicities (decrease in the body weights of the rats).

At the end of each BrdU labeling period (0 week delabed), we analyzed the percent of BrdU positive cells from the proliferative fraction of the crypt (where LRCs have previously been located). This maximal BrdU labeled sample was analyzed to confirm the efficacy of BrdU administration. There were 89, 65 and 77% BrdU+cells identified after weeks 2, 4, and 8 respectively, analyzed by FACS (77±12%, mean±SD).

Histograms of BrdU—Delabeled (Washed-Out) CEC

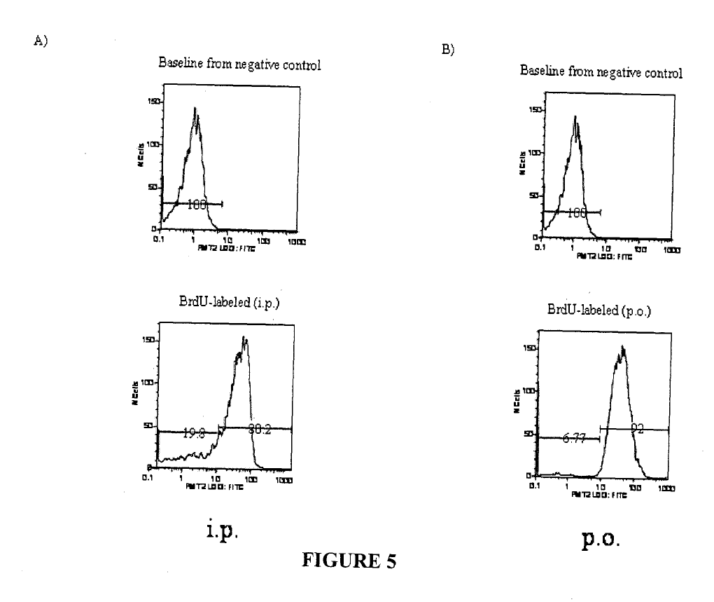

FIG. 5A shows the histograms of CEC from 2, 4 and 8 week BrdU delabeld rats. BrdU positive cells were collected using stringent criteria, selecting only the top 20-40% of the brighter end of the BrdU positive peak. FIG. 5B shows that there is no overlap between the BrdU positive and BrdU negative cells sorted by FACS, when BrdU positive and negative cells were reanalyzed by FACS after separation.

Percent of BrdU Positive Cells Measured by Flow Cytometry After Each Delabeling Period

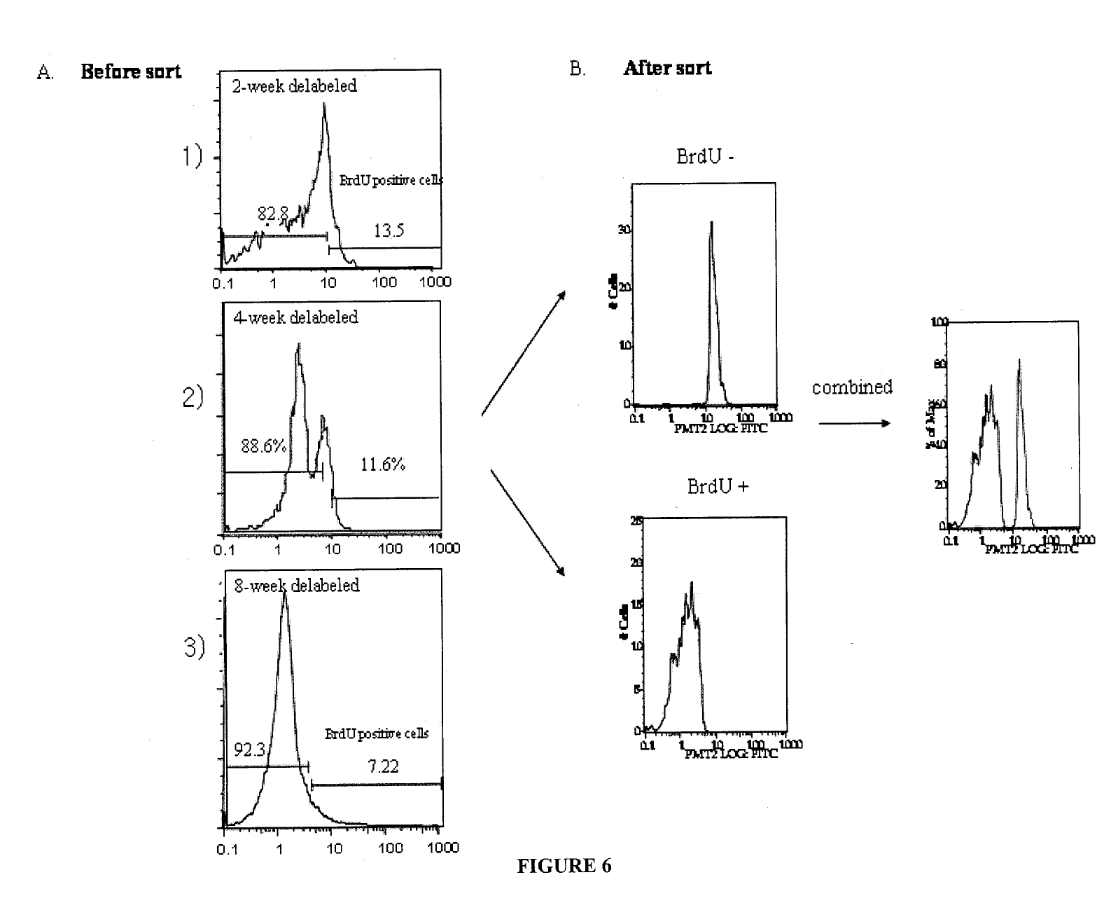

To determine the fraction of LRCs in the proliferative cell separation, percent of BrdU positive cells was measured by FACS during the BrdU delabeling period (FIG. 6). On average, there were 77, 12, 7.0 and 3.8% of LRC after 0, 2, 4 and 8 weeks of delabeling (4-9 animals/group), respectively (FIG. 6). This fraction of LRCs is similar to that reported by other researchers (6, 8, 17).

Proliferation Rates of Separated LRCs Using Deuterated Water

The LRCs were then collected by FACS for GC/MS analysis. The ²H enrichment of DNA from LRCs was measured by GC/MS and kinetics were calculated as described previously (20). Table 2 shows increase in ²H enrichments of LRC, reflecting LRC division during the ²H₂O labeling period after discontinuing BrdU intake. Among the total population of LRCs, an average of 0.33-0.9% divide per day or ˜0.8-2.5 million LRCs are newly produced/day, assuming the total number of cells in colon is ˜250 million. Average half-life of LRC was calculated to be 77-210 days.

TABLE 2
Kinetics of LRCs
Duration of BrdU-delabeling and ²H₂O administration

| | 2 weeks | 4 weeks | 8 weeks |
|---|---|---|---|
| f (%) | 12 | 15 | 17 |
| k (d⁻¹⁾ | 0.90 | 0.58 | 0.33 |
| t_{1/2 (days)} | 77 | 119 | 210 |

BrdU-delabeling and concurrent ²H²O labeling were carried out as described in the text (n = 22).

Percent of LRCs Using Deuterated Water Method

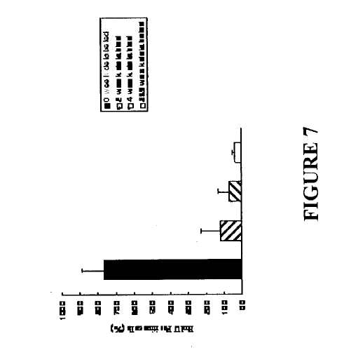

We also studied rats born after having been labeled in utero (the mothers had been on ²H₂O during the whole pregnancy). Three pups were on 4% ²H₂O for seven weeks after they were born and then were sacrificed (fully deuterated rats). The other four rats stopped ²H₂O intake at 7 weeks of age and were delabeled for 2 weeks (until ²H₂O had washed out of their body water pool) and then were sacrificed. The ²H enrichments between the fully deuterated and the delabeled CECs were compared. EM₊₁ were 10.53±0.2% and 1.04±0.18%, respectively, for fully labeled and 2 week delabeld animals. The EM₊₁ value of delabeled cells were 9.9% of that of fully deuterated CECs, which corresponds to the percent of LRCs in the delabeled rats (FIG. 7). It is similar to the percent of BrdU positive cells measured by FACS after 2 weeks of delabeling.

Location of LRC in the Colonic Crypt by BrdU Immunohistochemistry

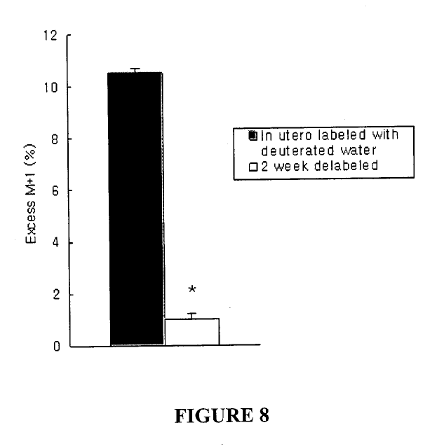

We determined the location of LRC by immunohistochemistry on 4 week BrdU delabeled crypts (FIG. 8B). In FIG. 8A, most of the CECs in fully BrdU labeled crypts after 2 week BrdU treatment were labeled brown, whereas in FIG. 8B, we could identify one cell that retained BrdU near the base of the crypt.

Aregentaffin Staining in Colon Crypts

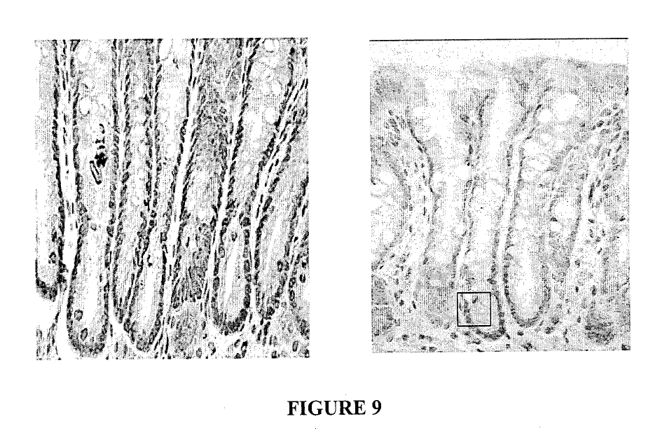

Since enteroendocrine cells, which is a minor population of the total epithelial cells (<1%, 28), are believed to have a longer lifespan (˜35-100 days) than the normal differentiating colonocytes, we determined the presence of enteroendocrine cells in our samples (FIG. 9). There was a negligible level of enteroendocrine cells present. Furthermore, they were not where LRCs are known to reside and identified in our study, which is mostly at the base of the crypts. Thus, we could effectively rule out significant contamination of this another type of long-lived cells in LRCs that we separated from colon.

The procedure for separating LRC from rat colon was labor intensive since LRCs are rare (3.8-12%) and we collected the upper 20-40% of the BrdU positive peak. Furthermore, CECs tend to aggregate, so we had to process nuclei on the FACS relatively slowly compared to blood cells; data processing rate of 400-500 events/sec vs 5000 events/sec). To collect 300,000-500,000 nuclei, the minimum number of nuclei needed for each GC/MS analysis, took 4-12 hours. Improvements in GC/MS techniques or instrumentation may reduce the cell number required for a mass spectrometric signal in future studies (Hellerstein, M K, Awada, M and Neese, R A, unpublished observations).

The relative ease and stability of the ²H enrichment in the body water is worth emphasizing. Stable body water enrichments were maintained throughout the study (4.2±0.2% of total body water, FIG. 3).

Results from the histograms from FACS confirmed the effective delivery of BrdU from drinking water to the colonocytes (FIGS. 4A,B). Approximately 90% of the CECs from the proliferative fraction were BrdU positive after 2 weeks of BrdU administration. However, the BrdU positive cells after 4 and 8 weeks of BrdU administration decreased to 65-77%. This reduction may be due to the induction of a liver enzyme that dehalogenates BrdU as a part of the animals' adaptation to the extended administration of BrdU (32). The induction of BrdU degrading enzyme makes BrdU less avalilable to be incorporated into cells. The drinking water method (p.o.) is the preferred method for prolonged administration of BrdU to i.p. injections due to the lower toxicity in the animals, but over time, apparently becomes less efficient as a label. Repeated i.p. injections of BrdU caused weight loss in rats (data not shown) although this was a more effective way of delivering BrdU to the colonocytes (FIG. 4).

The BrdU concentration used (1 mg/ml) for drinking water in this study is a commonly used concentration for long-term administration and did not cause any detectable toxicity or weight loss. It has been reported that 0.8 mg/ml of BrdU-containing drinking water administered for up to 5 weeks in B6 and BALB/C mice caused no thymic toxicity and 1 mg/ml of BrdU-containing drinking water given in LEW rats caused no apparent toxic effects on various fast-turning over tissues for up to 12 weeks. There have been reports on the inhibitory effects of BrdU on cell proliferation and differentiation in vitro and in vivo at various concentrations and in various cell types. Since BrdU toxicity is strain and cell type specific, we cannot completely assess possible toxic effects from BrdU on the proliferation of LRCs. But, since the LRC continued to divide during the delabeling period, based on the measured ²H incorporation into DNA from BrdU positive cells (Table 1) and the BrdU treated rats showed the same CEC proliferation rates as the control rats in our preliminary study (data not shown), we conclude that toxic effects are minimal.

The finding of BrdU positive cells after prolonged delabeling supports the existence of LRC in colon crypts (FIG. 5A). The percent of LRC after delabeling period of 2-8 weeks (FIG. 6) was 3.8-12%, which is in the range of the values previously reported. After sorting and collecting BrdU positive and negative nuclei, the collected nuclei were sorted again, separately, to confirm their difference in fluorescence intensity. The BrdU positive and negative nuclei formed peaks in different fluorescent regions and did not overlap (FIG. 5B).

Measurements of ²H enrichment in collected LRC allowed us to calculate the proliferation rates. There were 11.9±8, 14.9±6 and 16.9±8% newly synthesized cells based on ²H incorporation during ²H₂O labeling (during BrdU delabeling) for 2, 4 and 8 weeks, respectively. Using the exponential formulae, 0.5% of total LRC population divides on average each day.

The accuracy of the method was also supported by the study done with long-term administration of ²H₂O only. Deuterated water alone can be used for determining the presence of LRCs, although it does not allow separation of LRCs. Rats that were born after having been labeled in utero (dams had been placed on ²H₂O in drinking water before conception) should have all their CECs labeled with ²H, including LRCs and stem cells. Previously in our lab, we found it takes less than 1 week of delabeling for the ²H₂O to be almost completely washed out of the body water pool. Thus, the cells that still retain ²H after 2 weeks can be considered as LRCs. The percent of cells that still retained ²H after 2 weeks of delabeling was approximately 10% (FIG. 7), agreeing well with the results obtained with 2 week delabeling of BrdU.

We confirmed the effective labeling of CEC from the BrdU drinking water regimen by immunohistochemistry. Also, the location of LRC was identified by immunohistochemistry on BrdU delabled crypts. The LRC was at the base of the crypt, which is consistent with the location of colon stem cells identified with ³[H]-dT.

Enteroendocrine cells are known to comprise less than 1% of total CECs (28). We checked for the presence of this population that are reported to have a longer lifespan (35-100 days) than normal differentiating CECs. Using argentaffin staining, we were able to assess that the possible contamination from enteroendocrine cells in LRC was minimal (FIG. 9).

This safe in vivo method using D₂O is not only applicable to humans to measure colon epithelial cell proliferation without toxicity, but also can be used to determine adult stem cell proliferation rates.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit and scope of the appended claims.

Applicants do not abandon or dedicate to the public any unclaimed subject matter herein.

### REFERENCES

The following references cited herein are hereby incorporated by reference in their entirety:

1. American Cancer Society (ACS). (2002). Cancer facts and figures: Colbrectal Cancer and Early detection.

2. Wong, W. M. and Wright, N. A. (1999). Cell proliferation in gastrointestinal mucosa. J Clin Pathol., 52, 321-333.

3. Risio, M., Coverlizza, S., Ferrari, A., Candelaresi, G. L., and Rossini, F. P. (1988). Immunohistochemical study of epithelial cell proliferation in hyperplastic polyps, adenomas, and adenocarcinomas of the large bowel. Gastroenterology, 94, 899-906.

4. Terpstra, O. T., van Blankenstein, M., Dees, J. and Eilers, G. A. (1987). Abnormal pattern of cell proliferation in the entire colonic mucosa of patients with colon adenoma or cancer. Gastroenterology, 92,704-708

5. Morris, R. J. (2000). Keratinocyte stem cells: targets for cutaneous carcinogens. J Clin Invest 106, 3-8.

6. Bach, S. P., Renehan, A. G., and Potten, C. S. (2000). Stem cells: the intestinal stem cell as a paradigm. Carcinogenesis 21, 469-476.

7. Fearon, E. R. and B. Vogelstein, A. Genetic Model for Colorectal Tumorigenesis. Cell, 61: 759-768,1990.

8. Pozharisski, K. M., Klimashevski, V. F., and Gushchin, V. A. (1980). Study of kinetics of epithelial cell populations in normal tissues of the rat's intestines and in carcinogenesis. I. A comparison of enterocyte population kinetics in different segments of the small intestine and colon. Exp Pathol (Jena) 18, 387-406.

9. Bickenbach, J. R. (1981). Identification and behavior of label-retaining cells in oral mucosa and skin. J Dent Res 60 Spec No C, 1611-1620.

10. Cotsarelis, G., Sun, T. T., and Lavker, R. M. (1990). Label-retaining cells reside in the bulge area of pilosebaceous unit: implications for follicular stem cells, hair cycle, and skin carcinogenesis. Cell 61, 1329-1337.

11. Morris, R. J., Fischer, S. M., and Slaga, T. J. (1986). Evidence that a slowly cycling subpopulation of adult murine epidermal cells retains carcinogen. Cancer Res 46, 3061-3066.

12. Morris, R. J., Coulter, K., Tryson, K., and Steinberg, S. R. (1997). Evidence that cutaneous carcinogen-initiated epithelial cells from mice are quiescent rather than actively cycling. Cancer Res 57, 3436-3443.

13. Lipkin, M., Bell, B., and Sherlock, P. (1963). Cell proliferation kinetics in the gastrointestinal tract of man. I. Cell renewal in colon and rectum. J. Clin Investigation 42, 767-776.

14. Sunter, J. P., Wright, N. A., and Appleton, D. R. (1978). Cell population kinetics in the epithelium of the colon of the male rat. Virchows Arch B Cell Pathol 26, 275-87.

15. Potten, C. S., Li, Y. Q., O'Connor, P. J., and Winton, D. J. (1992). A possible explanation for the differential cancer incidence in the intestine, based on distribution of the cytotoxic effects of carcinogens in the murine large bowel. Carcinogenesis 13, 2305-2312.

16. Yatabe, Y., Tavare, S., and Shibata, D. (2001). Investigating stem cells in human colon by using methylation patterns. Proc Natl Acad Sci USA 98, 10839-10844.

17. Potten, C. S., Booth, C., and Pritchard, D. M. (1997). The intestinal epithelial stem cell: the mucosal governor. Int J Exp Pathol 78, 219-243.

18. Macallan, D. C., Fullerton, C. A., Neese, R. A., Haddock, K., Park, S. S., and Hellerstein, M. K. (1998). Measurement of cell proliferation by labeling of. DNA with stable isotope-labeled glucose: studies in vitro, in animals, and in humans. Proc Natl Acad Sci USA, 95, 708-713.

19. Neese, R. A., Siler, S. Q., Cesar, D., Antelo, F., Lee, D., Misell, L., Patel, K., Tehrani, S., Shah, P., and Hellerstein, M. K. (2001). Advances in the stable isotope-mass spectrometric measurement of DNA synthesis and cell proliferation. Anal Biochem, 298, 189-195.

20. Neese, R. A., Misell, L., Turner, S., Antelo, F., Kim, J., Cesar, D., Hoh, R., Chu A., Strawford, A., Christiansen, M., and Hellerstein, M. K. (2002) Measurement in vivo of proliferation rates of slow turnover cells by ²H₂O labeling of the deoxyribose moiety of DNA. PNAS.2002; 99:15345-15350.

21. Kim, J, Neese, R A, Isnard, P, McCune, M and Hellerstein, M K. A method to isolate long-term label retaining cells (putative stem cells) from rat colon of rats. (2002). Proceedings of American Association for Cancer Research, 127.

22. Brasitus, T. A., Isolation of proliferative epithelial cells from the rat cecum and proximal colon. (1982). Anal Biochem, 123(2), 364-372.

23. Brasitus, T. A. and R. S. Keresztes, Glycoprotein metabolism in rat colonic epithelial cell populations with different proliferative activities. (1983). Differentiation, 24(3), 239-44.

24.Todd, D., Singh, A. J., Greiner, D. L., Mordes, J. P., Rossini, A. A., and Bortell, R. A new isolation method for rat intraepithelial lymphocytes. (1999). J Immunol Methods, 224, 111-127.

25. Coles, M. C. and Raulet, D. H. NK1.1+ T cells in the liver arise in the thymus and are selected by interactions with class I molecules on CD4+CD8+ cells. (2000). J Immunol, 164, 2412-2418.

26. Murrill, W. B., Brown, N. M., Zhang, J. X., Manzolillo, P. A., Barnes, S., and Lamartiniere, C. A. Prepubertal genistein exposure suppresses mammary cancer and enhances gland differentiation in rats. Carcinogenesis, 17: 1451-1457, 1996.

27. Sheehan, D., and Hrapchak, B. (1980). Theory and practice of Histotechnology (Detroit, Mich.): Battelle Press, pp. 276-277.

28. Roth, K. A., and Gordon, J. I. (1990). Spatial differentiation of the intestinal epithelium: analysis of enteroendocrine cells containing immunoreactive serotonin, secretin, and substance P in normal and transgenic mice. Proc Natl Acad Sci USA 87, 6408-6412.

29. Deschner, E. E., and Lipkin, M. (1966). An autoradiographic study of the renewal of argentaffin cells in human rectal mucosa. Exp Cell Res 43, 661-665.

30. Kim, K. M., and Shibata, D. (2002). Methylation reveals a niche: stem cell succession in human colon crypts. Oncogene 21, 5441-5449.

31. Bjerknes, M. (1996). Expansion of mutant stem cell populations in the human colon. J Theor Biol 178, 381-385.

32. Jecker, P., Beuleke, A., Dressendorfer, I., Pabst, R., and Westermann, J. (1997). Long-term oral application of 5-bromo-2-deoxyuridine does not reliably label proliferating immune cells in the LEW rat. J Histochem Cytochem 45, 393-401.

33.Tough, D. F., and Sprent, J. (1994). Turnover of naive- and memory-phenotype T cells. J Exp Med 179, 1127-1135.

34. Goz, B. (1978). The effects of incorporation of 5-halogenated deoxyuridines into the DNA of eukaryotic cells. Pharmacol Rev 29, 249-272.

35. Rocha, B., Penit, C., Baron, C., Vasseur, F., Dautigny, N., and Freitas, A. A. (1990). Accumulation of bromodeoxyuridine-labeled cells in central and peripheral lymphoid organs: minimal estimates of production and turnover rates of mature lymphocytes. Eur J Immunol 20, 1697-1708.

36. Boman, B. M., Fields, J. Z., Bonham-Carter, O., and Runquist, O. A. (2001). Computer modeling implicates stem cell overproduction in colon cancer initiation. Cancer Res 61, 8408-8411.

37. Zhang T, Otevrel T, Gao Z, Ehrlich S M, Fields J Z, Boman, B M. Evidence that APC regulates surviving expression: a possible mechanism contributing to the stem cell origin of colon cancer. Cancer Res 2001:61:8664-8667.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit and scope of the appended claims.

We claim:

1. A method of separating one or more stem cells from a tissue or individual, said method comprising:
a) administering one or more cell-lineage-marking labels to said tissue or individual for a sufficient time for said one or more cell-lineage marking labels to incorporated into the cells of said tissue or individual;
b) discontinuing said administering step a) for sufficient time to form a first population of cells and a second population of cells wherein the cells of said second population of cells contain detectably more cell-lineage marking label than the cells of said first population of cells; and
c) detecting the presence or absence of said one or more cell-lineage marking labels in the cells of the first population of cells and the cells of the second population of cells; and
d) separating the cells of said second population of cells, wherein the cells of said second population of cells are stem cells.

2. The method of claim 1, wherein said first population of cells contains no detectable cell-lineage marking label.

3. The method of claim 1, wherein said cell-lineage-marking label is a halogenated deoxyribonucleotide.

4. The method of claim 3, wherein said halogenated deoxyribonucleotide is bromodeoxyuridine or iododeoxyuridine.

5. The method of claim 1, wherein said tissue is selected from the group consisting of colon, breast, small intestine, uterine cervix, prostate gland, skin, bone marrow, liver, heart, skeletal muscle, thymus, thyroid gland, pancreas, bladder, lung, biliary track, ovary, testes, brain, and lymphoid tissue.

6. The method of claim 1, wherein said cell-lineage marking label is detected by antibodies specific to said cell-lineage marking label.

7. The method of claim 1, wherein said separating step c) comprises fluorescence-activated cell sorting (FACS).

8. A method of identifying carcinogenesis in a tissue or individual, said method comprising:
a) separating one or more stem cells according to the method of claim 1, and
b) detecting a DNA modification in said one or more stem cells, wherein said DNA modification is associated with carcinogenesis.

9. The method according to claim 8, wherein said DNA modification is selected from the group consisting of a DNA chemical modification, a DNA cross-link, a DNA mutation, a base deletion, a base insertion and an intercalation.

10. The method of claim 8, further comprising correlating said DNA modification to a risk factor selected from the group consisting of carcinogen exposure, DNA repair capacity, oxidative damage and mutation risk.

11. A method of identifying a chemical agent as genotoxic, said method comprising the steps of:
a) administering said chemical agent to a tissue or individual,
b) separating one or more stem cells according to the method of claim 1, and
c) detecting DNA modification in said stem cells,
wherein said DNA modification in said stem cells identifies said chemical agent as genotoxic.

12. The method of claim 11, wherein said DNA modification is selected from the group consisting of DNA damage, DNA mutations and chemical alteration of DNA.

13. A method of identifying one or more phenotypic markers of stem cells, said method comprising:
a) separating one or more stem cells according to the method of claim 1, and
b) comparing phenotypes of said one or more stem cells to phenotypes of one or more non-stem cells to identify a difference in phenotypes between said one or more stem cells and said one or more non-stem cells, wherein said difference is a phenotypic marker of said one or more stem cells.

14. A method of determining the proliferation rate of one or more stem cells in a tissue or individual, said method comprising:
a) administering one or more cell-lineage-marking labels to said tissue or individual for a sufficient time for said one or more cell-lineage marking labels to incorporated in said tissue or individual;
b) administering one or more isotopically labeled DNA synthesis precursor to said tissue or individual;
c) discontinuing said administering step a) for sufficient time to form a first population of cells and a second population of cells wherein the cells of said second population of cells contain detectably more cell-lineage marking label than the cells of said first population of cells;
d) detecting the presence or absence of said one or more cell-lineage marking labels in said the cells of said first population and the cells of said second population;
e) separating the cells of said second population, wherein the cells of said second population are stem cells; and
f) measuring the isotopic enrichment of one or more deoxyribonucleotides in said stem cells to determine the proliferation rate of one or more stem cells in said tissue or individual.

15. The method of claim 14, wherein said first population of cells contains no detectable label.

16. The method of claim 14, wherein said one or more isotopically labeled DNA synthesis precursors is selected from the group consisting of ³H-dT, ²H-glucose and ²H₂O.

17. The method of claim 14, wherein said measuring step e) comprises measuring one or more hydrolysis products of said one or more deoxyribonucleotides.

18. The method of claim 17, wherein said one or more hydrolysis products are chemically modified.

19. The method of claim 14, wherein said measuring step comprises detecting said isotopic enrichment of said one or more deoxyribonucleotides by an analytic method selected from the group consisting of mass spectrometry, liquid scintillation counting, gamma counting, and nuclear magnetic resonance spectroscopy.

20. The method of claim 19, wherein said analytic method is mass spectrometry.

21. The method of claim 14, wherein said tissue is selected from the group consisting of colon, breast, small intestine, uterine cervix, prostate gland, skin, bone marrow, liver, heart, skeletal muscle, thymus, thyroid gland, pancreas, bladder, lung, biliary track, ovary, testes, brain, and lymphoid tissue.

22. The method of claim 14, further comprising calculating the clonal expansion factor of said second population of cells.

23. A method of identifying a therapeutic compound comprising:
a) administering a compound to a first group of one or more tissues or individuals;
b) determining the proliferation rate of one or more stem cells of said first group of one or more tissues or individuals according to the method of claim 14,
c) determining the proliferation rate of one or more stem cells of a second group of one or more tissues or individuals according to the method of claim 14, wherein said compound has not been administered to said second group of said one or more tissues or individuals; and
d) comparing the proliferation rate of said one or more stem cells of said first group to the proliferation rate of said one or more stem cells of said second group,
wherein a difference between the proliferation rate of the one or more stem cells of said first group and the one or more stem cells of said second group identifies said compound as a therapeutic compound.

24. The method of claim 23, wherein said compound is a chemical compound, dietary factor, or chemoprotective agent.

25. The method of claim 23, wherein said compound is a dietary factor.

26. The method of claim 25, wherein said dietary factor is selected from the group consisting of a soy-derived product, a brassica-derived product, and an anti-oxidant.

27. The method of claim 25, wherein said dietary factor is a soy-derived product selected from the group consisting of genistein and lunasin.

28. The method of claim 25, wherein said dietary factor is an anti-oxidant selected from the group consisting of vitamins C, vitamin E, and vitamin A.

29. A method of identifying a compound that stimulates stem cell proliferation, said method comprising:
a) administering a compound to a first group of one or more tissues or individuals;
b) determining the proliferation rate of one or more stem cells of said first group of one or more tissues or individuals according to the method of claim 14,
c) determining the proliferation rate of one or more stem cells of a second group of one or more tissues or individuals according to the method of claim 14, wherein said compound has not been administered to said second group of said one or more tissues or individuals; and
d) comparing the proliferation rate of said one or more stem cells of said first group to the proliferation rate of said one or more stem cells of said second group,
wherein an increase in the proliferation rate of the one or more stem cells of said first group over the proliferation rate of the one or more stem cells of said second group identifies said compound as a therapeutic compound.

30. The method of claim 29 wherein said compound is a putative lymphocyte co-stimulator and said tissue is one or more T cells.

31. The method of claim 14, further comprising measuring clonal or replicative exhaustion.

32. A method of identifying a disease or disorder by determining the proliferation rate of one or more stem cells according to the method of claim 14.

33. The method of claim 32 wherein said disease or disorder is diabetes mellitus and said tissue comprises one or more pancreatic β-cells.

34. The method of claim 32 wherein said tissue is one or more T lymphocytes and said disease or disorder is immune compromise.

35. The method of claim 32, wherein said immune compromise is a progressive lymphopenic disorders.

36. The method of claim 35, wherein said progressive lymphopenic disorder is acquired immune deficiency syndrome (AIDS).

37. The method of claim 32, wherein said tissue comprises one or more bone marrow cells and said disease or disorder is graft versus host disease (GVHD).

38. A kit for separating one or more stem cells in a tissue or individual comprising:
a) an cell-lineage-marking label, and
b) instructions for use of the kit,
wherein said kit is used to separate said one or more stem cells in a tissue or individual.

39. The kit of claim 38 further comprising an instrument for separating one or more stem cells from said tissue or individual.

40. The kit of claim 38 further comprising a tool for administering said cell-lineage-marking label.

41. A kit for determining the proliferation rate of one or more stem cells in a tissue or individual comprising:
a) an cell-lineage-marking label, and
b) an isotopically labeled DNA synthesis precursor,
wherein said kit is used to determine the proliferation rate of one or more stem cells in a tissue or individual.

42. The kit of claim 41 further comprising an instrument for separating one or more stem cells from said tissue or individual.

43. The kit of claim 41 further comprising an instrument for separating said one or more stem cells from said tissue or individual.

44. The kit of claim 41 further comprising a tool for administering said cell-lineage-marking label.

45. The kit of claim 41 further comprising a tool for administering said isotopically labeled DNA synthesis precursor.
